(19) United States
(12) Patent Application Publication
(10) Pub. No.: US 2020/0138312 A1
(43) Pub. Date: May 7, 2020

(54) NANOPATTERNED SURFACE MODIFICATIONS ON IMPLANTABLE MEDICAL DEVICE

(71) Applicant: Case Western Reserve University, Cleveland, OH (US)

(72) Inventors: Evon Ereifej, Cleveland, OH (US); Pamela Vandevord, Cleveland, OH (US); Jeffrey R. Capadona, Cleveland, OH (US)

(21) Appl. No.: 16/613,255

(22) PCT Filed: May 16, 2018
(86) PCT No.: PCT/US2018/032886

§ 371 Date: Nov. 13, 2019

Related U.S. Application Data
(60) Provisional application No. 62/507,493, filed on May 17, 2017.

(51) Int. Cl. A61B 5/04

(52) U.S. Cl. CPC A61B 5/04001; A61B 2562/0209

(57) ABSTRACT

The present disclosure relates to an implantable medical device with surface modifications to add additional surface area to the surface of the implantable device. The surface modifications create a rough, nanopatterned surface of the implantable medical device. The rough, nanopatterned surface can mimic a natural environment of an area of the subject's body, thereby reducing an immune foreign body response.

### RELATED APPLICATION

This application claims priority to U.S. Provisional Application Ser. No. 62/507,493, filed May 17, 2017, entitled “SURFACE MODIFICATIONS ON MEDICAL IMPLANTS/DEVICES”, the entirety of which is hereby incorporated by reference for all purposes.

### TECHNICAL FIELD

The present disclosure relates generally to implantable medical devices and, more specifically, to nanopatterned surface modifications on a surface of the implantable medical device.

### BACKGROUND

Traditionally, implantable medical devices, including neural implants, have had smooth and uniform surfaces to resist bacterial adhesion. While the smooth and uniform surface of neural implants does resist bacterial adhesion, implantation of neural implants causes a neural inflammatory response. Initially, the neural inflammatory response causes inflammation around the neural implant, but the neural inflammatory response leads to a chronic foreign body response. The chronic foreign body response can lead to encapsulation of the neural implant, which can cause reduced efficacy of the neural implant.

Current research aimed at alleviating the neural inflammatory response typically focuses on either therapeutic or materials-based solutions, such as electrode size, materials, protein and drug coatings, and/or administration. These solutions do not consider the native in vivo environment of the brain, which could be a key player in the neural inflammatory response. Within the brain, cells are living within the rough, textured environment of the extracellular matrix (ECM). The discontinuity between the rough, textured environment of the ECM and smooth surfaces of traditional neural implants may contribute to the initial inflammatory and chronic foreign body response to the implantation of the neural implants.

### SUMMARY

The present disclosure relates generally to implantable medical devices and, more specifically, to nanopatterned surface modifications on a surface of the implantable medical device.

In an aspect, the present disclosure can include a medical device. The medical device can include a three-dimensional structure with a rough, nanopatterned surface configured for implantation into an area of a subject's body. The rough, nanopatterned surface has an increased surface area compared to the smooth surface.

In another aspect, the present disclosure includes a method for manufacturing a medical device with nanopatterned surface modifications. The method includes the step of providing a medical device. The medical device includes a smooth surface configured to interface with an area of a subject's body. The method also includes modifying the smooth surface of the medical device to have a rough, nanopatterned surface has an increased surface area compared to the smooth surface.

In a further aspect, the present disclosure includes a method for using a medical device with nanopatterned surface modifications. The medical device is implanted into an area of a subject's body. The medical device includes a three-dimensional structure with a rough surface configured to interface with the area of the subject's body. The rough surface mimics a natural environment of the area of the subject's body to reduce an immune foreign body response.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a diagram showing an example of a system for creating surface modifications in a smooth surface of a medical device, according to an aspect of the present disclosure;

FIGS. 2-4 are diagrams showing examples of medical devices with surface modifications made by the system of FIG. 1;

FIG. 5 is a process flow diagram illustrating a method for using a medical device with nanopatterned surface modifications according to another aspect of the present disclosure;

FIG. 6 is a process flow diagram illustrating a method for making a medical device with nanopatterned surface modifications according to yet another aspect of the present disclosure;

FIGS. 7 and 8 show the effects of the nanopatterned implants on neuron viability compared to control implants;

FIGS. 9-12 show the effects of the nanopatterned implants on glial cell reactivity and inflammation compared to control implants;

FIGS. 13-15 show the effects of the nanopatterned implants on blood brain barrier permeability compared to control implants;

FIGS. 16-21 show a quantitative comparison between relative gene expression from tissue around nanpatterned implants and smooth control implants; and

FIGS. 22-29 show comparisons between the cellular and molecular tissue response around the nanopatterned side of the implant, around the non-nanopatterned side of the implant, and a control implant.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “medical device” refers to an instrument, apparatus, implement, machine, implant, or the like, which is intended for use in prevention, diagnosis, treatment, or cure of disease or other medical conditions.

As used herein, the term “implantable” refers to something that is capable of or designed for being implanted in living tissue. Accordingly, an implantable medical device is a medical device that is capable of or designed for being implanted in living tissue.

As used herein, the term “neural implant” refers to a type of medical device that is placed surgically in the nervous system, including the central nervous system (brain and spinal cord) and the peripheral nervous system. Examples of neural implants include a microelectrode, a spinal electrode, a sensor, a shunt, a cuff electrode, a percutaneous electrode, or the like.

As used herein, the term “surface” refers to a portion of a medical device that interfaces with a natural environment when implanted.

As used herein, the term “surface modification” refers to a physical change to the characteristics of a topography of a surface to achieve biomimetic functions. The surface modification can be accomplished by nanopatterning to cause physical changes to the nano-architecture of the surface. For example, the physical changes can be on a scale of 0.1-1000 nm. As another example, the physical changes can be on a scale of 0.5-500 nm. In a further example, the physical changes can be on a scale of 1-100 nm. Examples of nanopatterning can include nano-grooves, nano-pillars, nanofibers, or the like.

As used herein, the term “smooth” refers to having an even and regular nano-scale surface or consistency; free from perceptible projections, lumps, or indentations.

As used herein, the term “rough” refers to having an uneven or irregular nano-scale surface; not smooth or level.

As used herein, the term “etching” refers to the act of cutting or carving a pattern into a surface. The etching can be accomplished by ion beam, electron (e)-beam, electrochemical, direct imprint, thin coating, or the like.

As used herein, the term “natural environment” can refer to the environment into which the medical device is implanted. In many instances, the natural environment can include an extracellular matrix, which is generally a three dimensional meshwork of protein structures or proteins.

As used herein, the term “immune response” refers to the reaction of the cells and fluids of a patient's body to the presence of a substance that is not recognized as a constituent of the body itself. The immune response is a process including inflammation, wound healing, and/or end-stage tissue fibrosis.

As used herein the term “foreign body response” refers to the chronic, end-stage tissue fibrosis step of the immune response. In the case of an implantable medical device, the foreign body response includes encapsulation of the medical device, which can cause reduced efficacy of the medical device.

As used herein, the term “architecture” refers to the complex structure of something (e.g., the natural environment).

As used herein, the term “elastic modulus” refers to the ratio of the force exerted on something (e.g., the natural environment) to the resultant deformation.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

### II. Overview

The present disclosure relates generally to implantable medical devices.

Traditionally, medical devices, including neural implants, have a smooth surface structure. While the smooth structure is preferential for reducing bacteria growth, the smooth structure may contribute to an immune foreign body response, leading to encapsulation, which can cause reduced efficacy of the medical device. Accordingly, the present disclosure relates to reducing the immune foreign body response (both the initial inflammatory and the chronic foreign body response) by introducing nanopatterned surface modifications on a surface of the implantable medical device.

The nanopatterned surface modifications can be made directly into the surface (e.g., by etching). As opposed to other methods, like lithography, patterning, or other method that provides the modification to, but not within, the surface, etching provides a permanent modification that does not fall apart when the medical device is introduced into an area of a subject's body. The etching can cause physical changes to the nano-architecture of the surface to add more surface area to the surface of the medical device. The increased surface area provides a greater area for proteins to bind to and may alter protein conformation so that the cellular signaling may result in alterations in the cell morphology and phenotype. Additionally, the increased surface area can mimic a natural environment of an area of a subject's body upon implantation. In other words, the surface of the medical device can be altered from smooth to rough via the nanopatterned surface modifications.

For example, the area of the subject's body can have a rough, textured environment of the extracellular matrix (ECM). With the surface modifications, the nanopatterned surface of the medical instrument can mimic the rough, textured environment of the ECM, thereby reducing the discontinuity between the rough, textured environment of the ECM and smooth surfaces of traditional neural implants that may contribute to the initial inflammatory and chronic foreign body response.

### III. Systems

A typical medical device that is implanted within an area of a subject's body has a smooth and uniform surface to resist bacterial adhesion. While the smooth and uniform surface does resist bacterial adhesion, the smooth and uniform surface causes an immune response in the area of the subject's body where the medical device is implanted. This may be due to the in vivo environment of the area having a rough, textured character. The discontinuity between the smooth and uniform surface of the medical device and the rough, textured character of the environment of the area may contribute to the immune response.

To reduce the immune response, at least one rough, nanopatterned surface is added to the medical device. The rough, nanopatterned surface can be configured to interface with the area of the subject's body and mimic a natural environment of the area of the subject's body. The rough, nanopatterned surface can include one or more surface modifications that can alter a topology of the surface, an elastic modulus of the surface, or other aspect of the surface.

The rough, nanopatterned surface can cause physical changes to the nano-architecture of the surface to add more surface area to the surface of the medical device. Although reduction of the immune response will be described herein, the rough, nanopatterned surface can have additional affects, such as increasing the surface area of the medical device. The increased surface area provides a greater area for proteins to bind to and may alter protein conformation so that the cellular signaling may result in alterations in the cell morphology and phenotype.

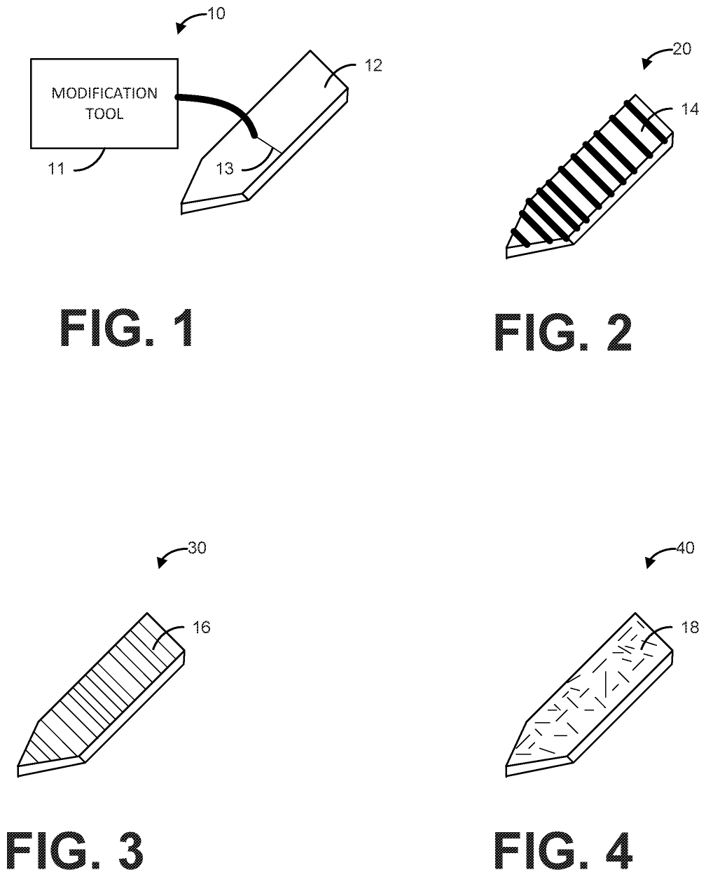

One aspect of the present disclosure can include a system 10 (FIG. 1) that can create the surface modifications 13 in the smooth surface 12 of the medical device. The surface modifications 13 can make the smooth surface into the rough, nanopatterned surface that can interface with the area of the subject's body. The surface modifications 13 address the discontinuity between the rough architecture of the tissue where the medical device will be implanted and the smooth surface 12. The discontinuity can contribute to the initial inflammatory and chronic foreign body immune responses that lead to a decreased efficacy of the medical device. As such, the surface modifications 13 created in the smooth surface 12 of the medical device increase the surface area of the medical device, thereby reducing negative consequences of the immune response.

The system 10 includes a modification tool 11 that can create the surface modifications 13. The surface modifications can be etchings into the surface of the medical device, and the modification tool 11 can use, for example, an ion beam, an electron (e)-beam, an electrochemical modality, a direct imprint modality, a thin coating modality, or the like to accomplish the etching. For example, the modification tool 11 can employ a gallium (Ga) ion beam to etch the surface modifications 13 into the smooth surface 12 of the medical device.

In some instances, the modification tool 11 can include a non-transitory memory to store a predefined pattern and a processor to execute the predefined pattern. The surface modifications 13 can be etched, by the modification tool 11, into the smooth surface 12 in the predefined pattern. For example, the program can allow the modification tool 11 to make the surface modifications 13 in an automated manner. In the automated execution of the program, the program can define certain coordinates (e.g., denoting the start and stop point for individual modifications, a depth of the modifications, a distance between the modifications, or the like) at which the surface modifications 13 are to occur. The coordinates can be different for different medical devices and/or for different areas in the subject's body where the medical device is destined to be implanted.

The predefined pattern can be selected from a plurality of predefined patterns based on the area of the subject's body into which the medical device will be implanted. Examples of medical devices 20, 30, 40 with surface modifications 14, 16, 18 in different patterns are shown in FIGS. 2-4. In FIG. 2, the surface modifications 14 are thick and either irregularly or regularly spaced. For example, the surface modifications 14 can be parallel lines 200 nm wide and 200 nm deep, spaced 300 nm apart. In FIG. 3, the surface modifications 16 are thin and either irregularly or regularly spaced. For example, the surface modifications 16 can be parallel lines 100 nm wide, 100 nm deep, spaced 100 nm apart. In FIG. 4, the surface modifications 18 are randomly oriented lines of different lengths, orientations, and spacings.

The pattern can be based on the nanoarchitecture of area of the subject's body where the medical device is destined to be implanted. For example, the area can be the central nervous system, and the medical device can be a microelectrode, a spinal electrode, or a shunt. As another example, the area can be the peripheral nervous system, and the medical device can be a cuff electrode or a percutaneous electrode. The central nervous system and the peripheral nervous system can have different characteristics of the nanoarchitecture. Even within the central nervous system, for example, the brain can have different characteristics of the nanoarchitecture than the spinal cord.

In some examples, the predefined pattern can be created and/or changed based on an input. The input can come from an external device (e.g., a remote computer, an input device keyboard, touchscreen, microphone, or the like) that is coupled to the modification tool 11. In some instances, the coupling can be via a wired connection. In other instances, the coupling can be via a wireless connection. In still other instances, the coupling can be via a connection that is both wired and wireless.

### IV. Methods

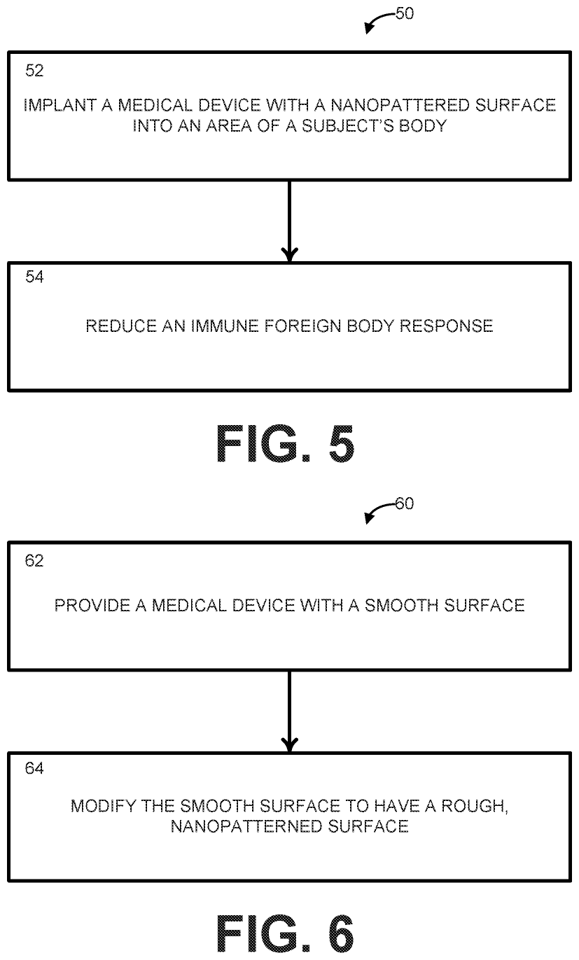

Another aspect of the present disclosure can include a method 50 (FIG. 5) for using a medical device with nanopatterned surface modifications (like the medical devices 20-40 shown in FIGS. 2-4 and described above). A further aspect of the present disclosure can include a method 60 (FIG. 6) for making a medical device with nanopatterned surface modifications.

The methods 50 and 60 are illustrated as process flow diagrams with flowchart illustrations. For purposes of simplicity, the methods 50 and 60 shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 50 and 60. However, additional aspects other than those illustrated may be required to implement the methods 50 and 60.

Referring now to FIG. 5, illustrated a method 50 for using a medical device with nanopatterned surface modifications. At step 52, a medical device with a nanopatterned surface can be implanted within an area of a subject's body. The medial device can include a three-dimensional structure with a rough surface configured to interface with the area of the subject's body. The rough surface can include etchings into the smooth surface of the medical device. The rough surface mimics a natural environment of the area of the subject's body so that, at step 54, an immune foreign body response in the area is reduced. For example, the natural environment of the area can include a three dimensional meshwork of proteins, and the topology of the modified medical device can include the nanopatterned surface that mimics the three dimensional meshwork of protein structures

Additionally or alternatively, the rough, nanopatterned surface can cause physical changes to the nano-architecture of the surface to add more surface area to the surface of the medical device. Although reduction of the immune response is one example, the rough, nanopatterned surface can have additional affects, such as increasing the surface area of the medical device. The increased surface area provides a greater area for proteins to bind to and may alter protein conformation so that the cellular signaling may result in alterations in the cell morphology and phenotype.

For example, the area of the subject's body can include neural tissue. In some instances, the area of the neural tissue can be the central nervous system. The medical device can be implanted into an area of the extracellular matrix (ECM) surrounding the neural tissue. The medical device can be a microelectrode, a spinal electrode, a shunt, a cuff electrode, a percutaneous electrode, or the like.

Referring now to FIG. 6, illustrated is a method 60 for making a medical device with nanopatterned surface modifications. The medical device can be nanopatterned by the system 10 of FIG. 1. The medical device can be configured for implantation into an area of a subject's body. The method 60 can be executed by the system 10 for creating surface modifications in a smooth surface of a medical device shown in FIG. 1.

At 62, a medical device can be provided with a smooth surface. The medical device can be configured to interface with an area of the subject's body. At 64, the smooth surface can be modified to have a rough, nanopatterned surface. The modification can be via etching. At least one side of the surface can be modified. As an example, the side of the surface that is modified can be configured to interface with the area of the subject's body. The rough, nanopatterned surface mimics a natural environment of the area of the subject's body. As an example, the rough, nanopatterned surface can include a surface modification that alters a topology of the surface, an elastic modulus of the surface, or the like. The nanopatterned surface is designed to reduce an immunological foreign body response within the area of the subject's body. As another example, the rough, nanopatterned surface can cause physical changes to the nano-architecture of the surface to add more surface area to the surface of the medical device. Although reduction of the immune response will be described herein, the rough, nanopatterned surface can have additional affects, such as increasing the surface area of the medical device. The increased surface area provides a greater area for proteins to bind to and may alter protein conformation so that the cellular signaling may result in alterations in the cell morphology and phenotype.

### V. Experimental

Traditionally, a medical device, like intracortical microelectrodes, includes a smooth surface. However, this smoothness creates a discontinuity between the rough architecture of the tissue and smooth device, which may contribute to the initial inflammatory and chronic foreign body response to the implantation of the medical device. The following experiment investigates the effects of nano-architecture surface modifications etched into the smooth surface of a traditional medical device on the initial inflammatory and chronic foreign body response to the implantation of the medical device. The following experiment is shown for the purpose of illustration only and is not intended to limit the scope of the appended claims.

### Methods

### Fabrication of Topographical Etchings

Nanopatterned surface modifications were fabricated onto non-functional Michigan-style silicon microelectrode neural probes. Standard photolithography methods were used on silicon on insulator (SOI) wafers to custom-made, non-functional, Michigan-style silicon microelectrode neural probes. Briefly, photoresist was spin-coated onto SOI wafers with a 15 μm-thick functional layer and up to a 3 μm-thick buried oxide. Deep reactive ion etching (DRIE) was used to shape probes 2 mm in length, 123 μm wide. The 15 μm-thick sheet of probes was released from the bulk silicon by use of hyrdofluoric acid, and photoresist was washed away in acetone. Each of the 1000 probes on the final silicon piece was held in place by a patterned mounting tab until being mechanically released for use. Neural probes were individually cut off the wafer, cleaned with three washes of 95% ethanol for five minutes each and allowed to air dry on a Teflon plate. The clean probes were mounted with silver paint onto an aluminum stand in the SEM in order to anchor the microelectrode probe tab away from the area that was going to be etched (the microelectrode probe shank). A FEI Helios 650 Field Emission Scanning Electron Microscope (SEM) with Focused Ion Beam (FIB) (Swagelok Center for Surface Analysis of Materials (SCSAM) at Case Western Reserve University) was utilized to etch the surface modifications on the neural probes. FEI Nanobuilder software was utilized to write an automated process that etched the specific nanopattern onto the silicon surface of the probes. Once the probes were focused and aligned properly, a gallium ion beam was used to etch the silicon and create parallel lines across the shank of the neural probe. Only one side of the probe was nanopattern surface modified in this study. The final dimensions of the etched lines were 200 nm wide and spaced 300 nm apart and were 200 nm deep. In preparation for surgery, surface modified neural probes and control non-surface modified silicon probes were cleaned with three 95% ethanol washes for five minutes per wash and one deionized water wash for five minutes. Subsequently, implants were sterilized using the following ethylene oxide gas protocol: 54.4° F., 1 hour sterile time and 12 hours aerate. By using ethylene oxide sterilization, implants fall within the FDA guidelines of maximum residual endotoxin concentration for cerebral spinal fluid contacting implanted devices (<0.06 EU mL-1).

### Neural Probe Implantation Procedure

The Institutional Animal Care and Use Committee (IACUC) at the Louis Stokes Cleveland Department of Veterans Affairs Medical Center approved all animal procedures. A total of seventeen (17) adult male Sprague Dawley rats (8-10 weeks old, ˜225 m) were used in this study. Animals were implanted with neural probes in the sensory cortex for either two or four weeks. Nanopatterned surface modified neural probes were compared to non-patterned control probes. Four animals were used in the nanopattern group at 2 weeks, four animals were used in the nanopattern group at 4 weeks, five animals were used in the control group at 2 weeks, and four animals were used in the control group at 4 weeks. The same animals were used for histological and genomic analysis. Rats were anesthetized in an isoflurane chamber (3% in 1.5 L/min O₂) for four minutes. Once anesthetized to the surgical plane, anesthesia was maintained with isoflurane at 2.5% using a nose cone. The animals' heads around the surgical site was shaved, and Marcaine was administered subcutaneously (SQ) around the surgical site. Carprofen (5 mg/kg) and Cefazolin (25 mg/kg) were administered SQ for analgesic and antibiotics respectively. The animal was mounted onto a stereotaxic frame with 1-2.5% isoflurane flowing through the nosecone for maintenance of anesthesia. The surgical site was sterilized with alternating cotton tipped applicators of chlorhexidine gluconate (CHG) and isopropanol scrubs. Animal body temperature was maintained on a circulating water pad and vitals (body temperature, heart and respiratory rate, and oxygen levels) were consistently monitored using a MouseSTAT® Pulse Oximeter & Heart Rate Monitor, (Kent Scientific Corp., Torrington, Conn.).

An incision of the scalp was made along the midline and the skin was retracted to view the skull. The periosteum was cleaned off the skull using cotton tipped applicators and gauze. Following which, hydrogen peroxide was applied to the skull surface using a cotton tipped applicator in order to dehydrate the skull. The skull was then primed using Vetbond animal tissue adhesive. Using a sterile ruler and forceps, the area to be drilled was marked, 2 mm lateral to midline and 3 mm posterior to bregma (sensory cortex). Using a 2 mm drill bit, the skull was drilled, exposing the dura. A fine 45° angle dura pick was used to reflect the dura and uncover the brain. Implants were inserted into the cortex, approximately 2 mm deep, by hand, avoiding vasculature. The direction of which the nanopattern side was inserted was carefully noted for future correlation between nanoarchitecture and histological markers or RT-PCR analysis of gene expression. Kwik-sil (World Precision Instruments, Sarasota, Fla.) was applied over the implant site to keep the brain insulated. Fusio and Flow-it ALC (Pentron Clinical, Wallingford, Conn.) UV-cured dental cement were used to build a stable headcap covering the implant hole and tab. The skin was pulled together and sutured closed with 5-0 monofilament polypropylene suture (Henry Schein, Melville, N.Y.), and covered with triple antibiotic ointment. Analgesia and antibiotics were administered for three days post-operatively following surgery.

### Tissue Processing

At the pre-determined end points (2 or 4 weeks post implantation), animals were anesthetized with intraperitoneal (IP) injections of ketamine (160 mg/kg) and xylazine (20 mg/kg). Animals were perfused transcardially with 1× Phosphate Buffer Saline (PBS, Invitrogen, Carlsbad, Calif.) until the body was clear of blood and then equilibrated with 30% sucrose (Sigma, St. Louis, Mo.) in 1×PBS. The brain was gently removed from the skull and the microelectrode probe was carefully removed from the brain. The brain was then frozen in optimal cutting temperature compound (OCT, Tissue Tek, Torrance, Calif.) on dry ice and then transferred to a −80° C. freezer to be cryosectioned. Prior to cryosectioning, the cryostat, blades and slides were cleaned with RNaseZap (Thermo Fisher Scientific, Waltham, Mass.) an RNase decontamination solution to remove any RNase enzymes. The brains were sliced transversely in 20 μm sections at −21° C. and mounted onto either glass slides for staining or Leica FrameSlides PEN-Membrane 4.0 μm (Leica, Wetzlar, Germany) slides for Laser Capture Microdissection and downstream genetic analysis. Slides were prepared during cryosectioning to contain one slice every 650 μm along the depth of the probe shank. The slides were stored at −80° C. until LCM or immunohistochemistry labelling.

### Histology

The localized tissue reaction for activated microglia, blood-brain barrier permeability, astrocytes and total neurons was investigated. Two slides, each with three randomly selected tissue slices from the cortex, were selected to be stained from each animal. There were six tissue slices per animal, at every time point for each group stained with each cellular marker (total 24-30 tissue slices/stain per animal). Briefly, frozen slides were equilibrated to room temperature (RT) for one hour, and OCT was removed with three 1×PBS washes. Tissue was then fixed with 4% formaldehyde for ten minutes at RT, followed by six washes of 1×PBS containing 0.1% Triton-X 100 (Sigma, St. Louis, Mo.) (1×PBS-T) to rehydrate and permeabilize the tissue. The tissue was blocked for one hour at RT with goat serum blocking buffer (4% v/v serum (Invitrogen, Carlsbad, Calif.), 0.3% v/v Triton-X 100, 0.1% w/v sodium azide (Sigma, St. Louis, Mo.). Following which, the tissue was incubated overnight at 4° C. with primary antibodies diluted in goat serum blocking buffer. Primary antibodies used were: mouse anti-neuronal nuclei (NeuN) (1:250, Millipore, Billerica, Mass.) for neurons, rabbit anti-glial fibrillary acidic protein (GFAP) (1:500, Dako, Santa Clara, Calif.) for astrocytes, mouse anti-CD68 (ED1) (1:100, Millipore, Billerica, Mass.) for activated microglia/macrophages, and rabbit anti-immunoglobulin G (IgG) (1:100, AbD Serotec, Hercules, Calif.) for blood brain barrier permeability. NeuN and IgG were co-stained together on the same tissue, while CD68 and GFAP were co-stained together. After eighteen hours incubation, the tissue was washed six times with 1×PBS-T for five minutes per wash to remove any unbound primary antibodies. Alexa Flour conjugated secondary antibodies (diluted 1:1000 in blocking buffer) was incubated for two hours at RT along with 4′,6-diamidino-2-phenylindole (1:3600, DAPI, Thermo Fisher Scientific, Waltham, Mass.) to counterstain all cell nuclei. The tissue was washed six times with 1×PBS-T for five minutes per wash, followed by a ten minute incubation in 0.5 mM copper sulfate buffer (50 mM Ammonium Acetate, pH 5.0) (Sigma, St. Louis, Mo.) to remove tissue autofluorescence. Samples were rinsed with distilled water and mounted with Fluoromount-G (Southern Biotech, Birmingham, Ala.). Slides were imaged using a 10× objective on an inverted Carl Zeiss AxioObserver Z1 (Carl Zeiss Inc., Thornwood, N.Y.) and AxioCam MRm monochrome camera (Carl Zeiss Inc., Thornwood, N.Y.). In order to image the entire area of interest around the implant site, the MosaiX module in the AxioVision LE software was utilized to take sixteen tile images and then stitch them together into one final image. The exposure times were optimized and held constant for all images taken for each cellular marker analyzed. Unaltered, linearized images were exported as 16-bit tagged imaging files (TIFs) for quantitative analysis.

### Explanted Probe Staining and Imaging

Explanted probes were placed into microcentrifuge tubes containing 1 mL of 4% formaldehyde to fix any adhered cells and then stored at 4° C. for later staining. The day of staining, the probes were carefully removed from the tubes and placed onto glass coverslips in a 6 well dish. Explanted probes were washed six times with 1×PBS-T to permeabilize the cell membrane, followed by incubation with DAPI (1:3600, Thermo Fisher Scientific, Waltham, Mass.) for 2 hours at RT to stain cell nuclei. The probes were then washed six times with 1×PBS and mounted onto glass slides for imaging. Explanted probes were imaged using an inverted Keyence BZ microscope (Osaka Prefecture, Japan) at 100× magnification. Following which, the same probes were imaged using the FEI Helios 650 Field Emission Scanning Electron Microscope (SEM). The non-patterned side of the probes were imaged using 1 kV acceleration voltage and the patterned side required 5 kV in order to image the nanopatterns. All samples were imaged using 100 pA current for the lower magnification (<2500×). To obtain better high resolution images at higher magnification (>2500×), images were taken with TLD (Through Lens Detector) mode 2 using 1 kV 13 pA.

### Quantitative Analysis

Following image acquisition, all cellular markers except for NeuN were analyzed using SECOND, a custom MATLAB program developed to analyze fluorescent intensity profiles around the microelectrode probe. To quantify neuron populations around the implant site, the number of neurons were manually counted using a custom subset of the SECOND MATLAB code. Tiff images of the cellular marker of interest, DAPI and bright field were uploaded into SECOND, and the implant site was manually defined and marked utilizing the bright field image. The program then defined bins, each consisting of 5 μm wide concentric rings, spanning out to 1 mm from the implant site (0 μm is defined as the edge of the hole). Raw fluorescent intensity quantification from each tissue section of activated microglia, astrocytes and blood brain barrier permeability were normalized to background, defined as the average fluorescent intensity from 700-750 μm from the implant site. After normalization, the area under the curve from 0 to 50, 50 to 100, 100 to 150, and 150 to 200 μm were obtained using MATLAB and used in statistical analysis. The area under the curve is the trapezoidal approximation of binning the normalized fluorescent intensities of distance intervals of interest. The use of AUC allows us to perform statistical analysis on the distance intervals of interest, as opposed to individual fluorescent points.

The images from the animals that received surface modified implants were evaluated one semi-circle at a time, in order to analyze the circumferential effects of the nanopattern. The MATLAB code was run twice per image: 1) the half of the tissue facing the nanopatterned side of the probe and 2) the half of the tissue facing the non-patterned side of the probe. The same 5 μm wide concentric rings were drawn around the implant site and the fluorescent intensity was quantified. The areas under the curve from each semi-circles was compared to one another in statistical analysis.

To quantify neurons around the implant site, the implant site was manually defined in SECOND. Concentric rings were defined in the program up to 500 μm away from the implant site. The number of neurons were manually counted to obtain the number of neurons per area for each radial distance. The number of neurons counted in the ring 450-500 μm away from the implant site was utilized as the background value to be normalized against, ensuring that the normalized density was consistent within a cortical layer. Neuron counts were then converted to percentages to the background value.

### Laser Capture Microdissection

Tissues, frozen in blocks of OTC were sectioned onto Leica frame slides (membrane thickness 4 μm) and kept at −80° C. until further use. There were 18 tissue slices per animal, at every time point used for LCM tissue collection. On the day scheduled for LCM, slides were removed from −80° C. and without thawing, immediately prepared for staining by submerging in an ethanol series: 95% (30 s), 70% (30 s), 50% (30 s). Cresyl Violet (in 50% ethanol) was used to stain the tissue, followed by dehydration series according to the kit manufacturer's protocol (AM1935, Ambion, Waltham, Mass.). Finally, slides were immersed in xylene (5 min) and air-dried (5 min). Slides were then taken to an RNase contamination-free Leica LMD7000 microdissection system. Implant sites were identified on the LCM microscope. For control implants, circles (500 μm diam.) centered on the hole were drawn around the implant site using Leica LMD software. The tissue that had the surface modified implants were similarly defined with circles radiating 500 μm away from the implant site. However based on the direction the surface modified side of the microelectrode probe was facing, the tissue was cut in half and collected separately. Upon microdissection, the cut pieces of tissue fell directly into 500 μL tubes containing an RNA extraction lysis buffer, Qiazol (Qiagen, Valencia, Calif.). The collected tissue in Qiazol was kept on ice until all tissue of interest was collected for that day. RNA was extracted and purified the same day of collection and stored at −80° C. for further processing.

### Real Time Polymerase Chain Reaction

RNA was purified using RNeasy Micro Kit (Qiagen, Valencia, Calif.) following the manufacturer's protocol. The RNA concentration and purity was determined using a NanoDrop (Thermo Fisher Scientific, Waltham, Mass.). Reverse transcriptase using random primers converted the mRNA to a cDNA template using a thermal cycler (GeneAmp PCR System 9700, Applied Biosystems, Foster City, Calif.) and following the manufacturer's (Qiagen RT² Profiler, Qiagen, Valencia, Calif.) protocol. For PCR analysis, a cDNA equivalent to 40 ng of total RNA was used. Custom RT² Profiler PCR Arrays (Qiagen, Valencia, Calif.) were designed to include specific genes of interest for astrocyte activation (GFAP), inflammatory cytokines (IL1β and TNFα), oxidative stress marker (NOS2), necrosis marker (HMGB1), and inflammatory receptor (CD14). Each custom plate contained positive PCR controls, reverse transcriptase controls, genomic DNA contamination controls, as well as three endogenous controls, actin beta, beta-2 microglobulin and tata box binding protein. The tata box binding protein (TBP) was utilized as the endogenous control. SYBR green (Qiagen, Valencia, Calif.) was utilized as the fluorescence tag. cDNA templates and master mix were read in a 96-well optical plate using a 7900HT Real-Time PCR System (Applied Biosystems, Foster City, Calif.) running the following protocol: 1) Hold 95° C. for 10 min 2) 40 Cycles at 95° C. for 15 sec and 60° C. for 1 min. Melt curves for each gene were ran and evaluated to verify proper runs running the following: 1) Hold 95° C. for 15 sec 2) Hold 60° C. for 15 sec 3) Hold 95° C. for 15 sec. Threshold cycle (Ct) values for each sample and primer pair were obtained and analyzed through the SDS 2.3 software (Applied Biosystems, Foster City, Calif.) with the delta (Δ) Ct method in order to calculate the relative gene expression fold change (R).

The following equations were used:

ΔCt=Ct(Gene of Interest)−Ct(TBP);

R=2^{ΔCt}.

### Statistical Analysis

For statistical analysis, the area under the curve for all stains (except for NeuN) was used. For NeuN, the number of neurons per area was used for analysis. Statistical analyses was performed using a 2×2 factorial model in Minitab 16 (Minitab Inc., State College, Pa.) to allow for comparisons between conditions. There were six slices from different depths of the brain used from each animal in order to obtain data across the length of the implant. All images were averaged with the entire group of images from a single animal. For significance, t-tests of the effect estimates were performed and significance was defined as p<0.05. Gene expression analysis was performed using t-test in Minitab. All of the RNA extracted from one animal was pooled together and treated as an independent sample. Significance was defined as p<0.05.

### Results

The following results demonstrate that nanopatterned surface modifications etched into an intracortical microelectrode probe reduce the neuroinflammation seen after intracortical microelectrode probe implantation.

Feasibility of Etching Nanopatterned Grooves onto Microelectrode Probe Surfaces

Non-functional Michigan-style silicon shank microelectrode probes were nanopatterned on one side, using focused ion beam (FIB) etching to create nanoscale parallel grooves on the device surface. A gallium ion beam was utilized to etch silicon away and create parallel grooves across the shank of the microelectrode probe. FEI Nanobuilder software was utilized to write an automated program that etched the specific nanopattern onto the silicon surface of the probes. The final dimensions of the etched lines were 200 nm wide and spaced 300 nm apart and were 200 nm deep.

It must be highlighted that the in vitro studies utilized polymers, such as poly(methyl methacrylate) (PMMA) and polydimethylsiloxane (PDMS). These polymers both have an elastic modulus closer to that of the brain tissue thus rendering them more biocompatible as neural implants. The elastic modulus of the brain is between 0.6-180 kPa, PDMS has a modulus ranging between 100 kPa and 3.7 MPa, and PMMA has a modulus of 1.8-3.1 GPa. Whereas the modulus of the silicon used in this in vivo study is 70 GPa. While the influence of implant stiffness has been demonstrated by several groups, stiffness is not the only influencer of the neuroinflammatory response to intracortical microelectrode probes, and thus the relative contribution between one factor over the other cannot be determined. Here, for the first in vivo investigation of nanopatterned grooves, silicon nonfunctional Michigan style microelectrode probes were used for consistency to what is readily available to the field.

### Effects of Nanopatterned Grooves on Neuron Viability

In order to record the neural activity of single neurons, the soma must be within 50-140 μm from the microelectrode recording site. The presence of neuroinflammation and glial cell activation can directly alter neuronal populations. Nanopatterned surface modifications have been shown to reduce glial cell activation in vitro. Therefore, here it was investigated if nanopatterned grooves etched onto the surface of the intracortical microelectrode could affect the neuronal density immediately around implanted microelectrodes. To understand the viability of the neurons at the microelectrode-tissue interface, investigation of the population of labeled neurons expressed NeuN, a neuronal nuclei marker, was performed. All neuron counts are presented as a percentage of a background region from the same animals. For all histology, the “nanopatterned” microelectrode included both sides of the shank in analysis, and were compared to control, “smooth” microelectrodes, which also included both sides of the shank.

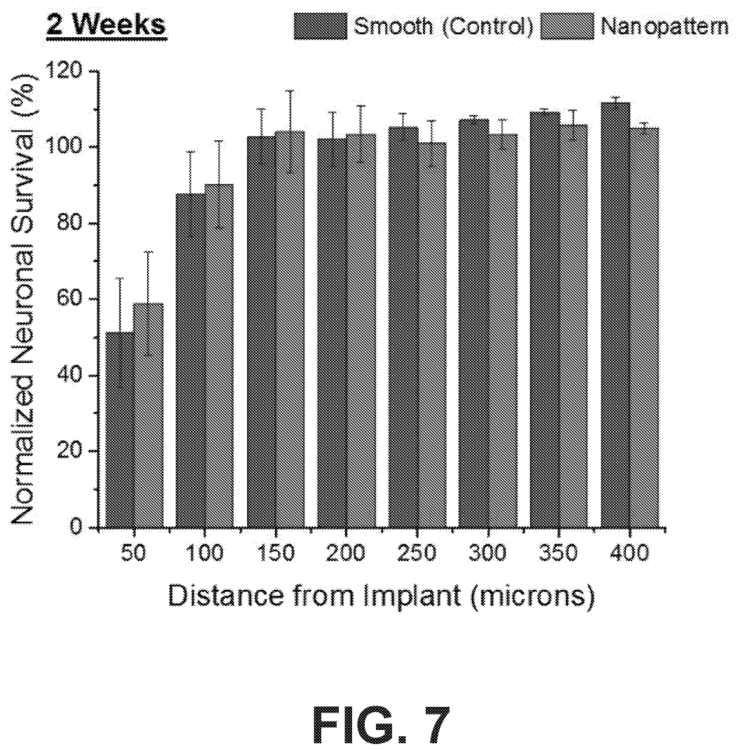

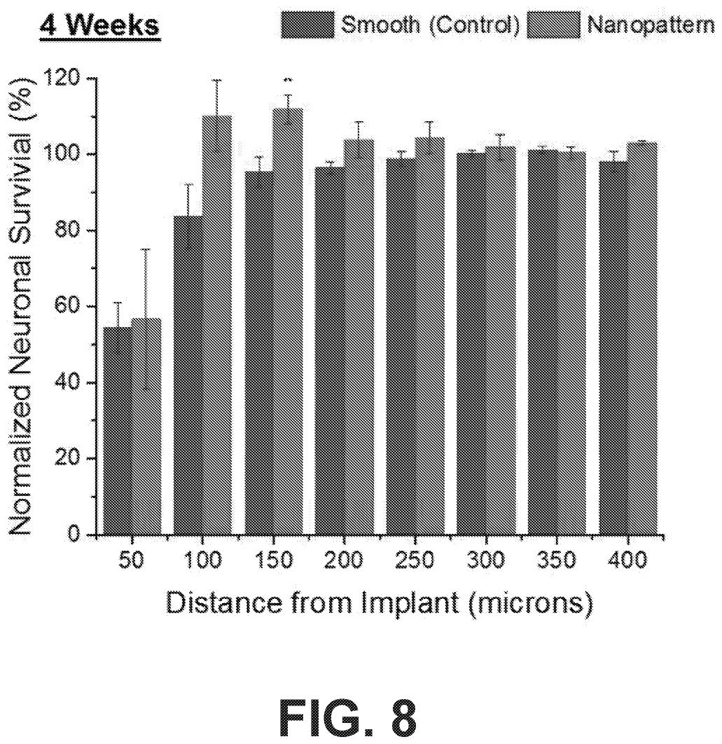

No significant differences in neuron densities were found at two weeks post-implantation between nanopatterned and smooth control implants (FIG. 7). However, there were significantly more neurons at the 100-150 μm distance around nanopatterned implants observed at four weeks post-implantation compared to the smooth control implants (p<0.05 vs. controls) (FIG. 8). There is a direct correlation between the neuroinflammatory response, the proximity of neurons to the microelectrode, and the ability to maintain quality intracortical recordings from microelectrodes. Furthermore, the neuronal density increased around nanopatterned implants over time contrary to the observed decrease in neuronal density found around control implants. Although this observation was not statistically significant, it may suggest that the use of nanopatterned grooves onto microelectrodes may translate into improved recording quality and stability in future studies.

### Effects of Nanopatterned Grooves on Glial Cell Reactivity and Blood Brain Barrier Permeability

To investigate the effect nanopatterned grooves etched onto the surface of the intracortical microelectrodes have on glial cell activation, inflammation and glial scarring, immunohistochemistry (IHC) staining was performed. Astrocytes are known to form a dense scar encapsulating the electrode and surrounding activated microglia after device implantation. Furthermore, activated microglia and macrophages release pro-inflammatory markers, neurotoxic agents and reactive oxygen species that have been linked to the neuroinflammation observed after intracortical microelectrode implantation. Astrocytes were labelled with glial fibrillary acidic protein (GFAP) antibody, an astrocyte specific marker that stains the intermediate filaments of immature/mature resting or activated astrocytes. GFAP is upregulated in a proinflammatory phenotype, compared to a quiescent phenotype. Additionally, activated microglia/macrophages were labelled with CD68, a highly expressed cytoplasmic antigen found only in activated microglia/macrophages.

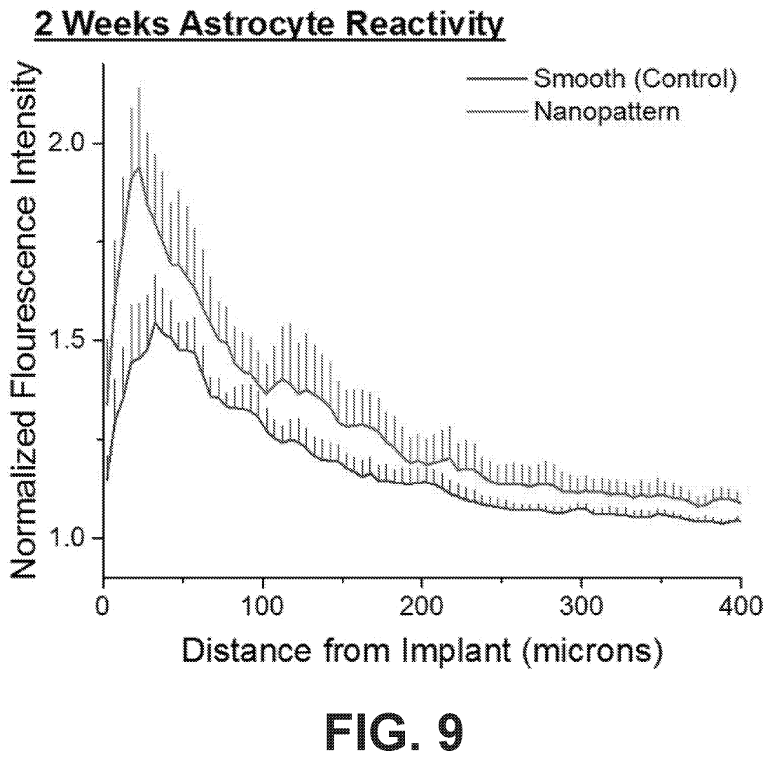

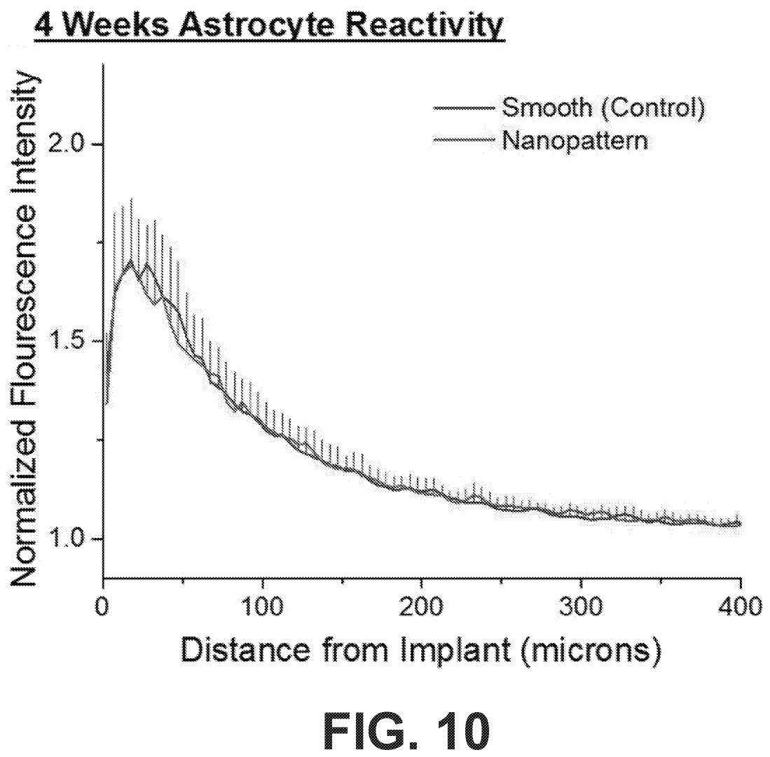

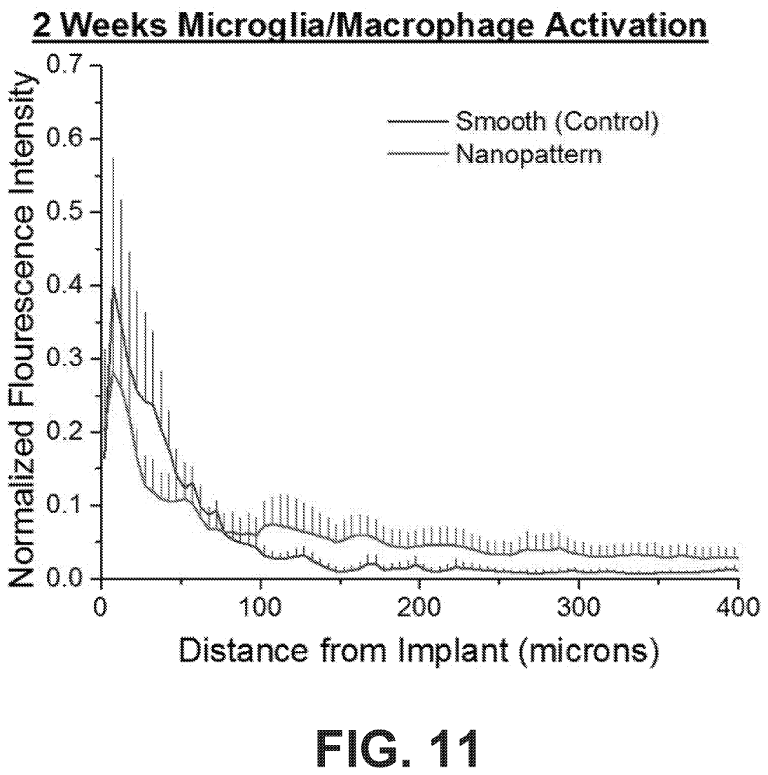

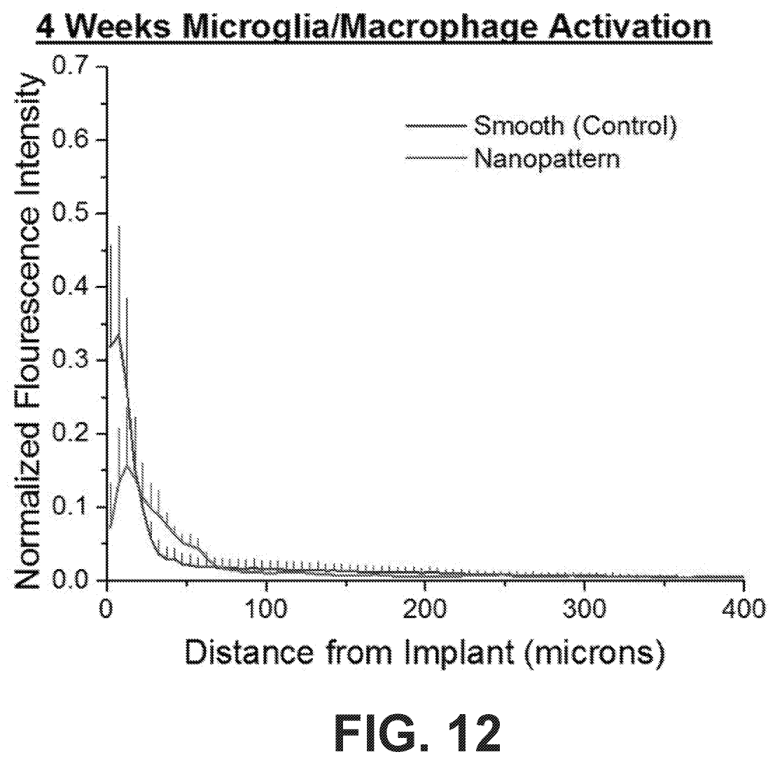

At two and four weeks post-implantation, the density of astrocytes and level of astrogliosis around the implant site were not significantly different between nanopatterned and smooth control animals (FIGS. 9, 10). Similarly, at both two and four weeks post-implantation, the density of activated microglia/macrophages around between nanopatterned and smooth control implant sites were not significantly different (FIGS. 11, 12).

Intracortical microelectrode implantation causes disruption of the blood brain barrier (BBB) and infiltration of blood derived cells and serum proteins into the brain tissue. Chronic BBB breach has been correlated with neuroinflammation and decreased microelectrode function. In order to assess the integrity of the blood brain barrier over time after neural probe implantation, IHC was performed staining for IgG, a serum antibody only found in the blood.

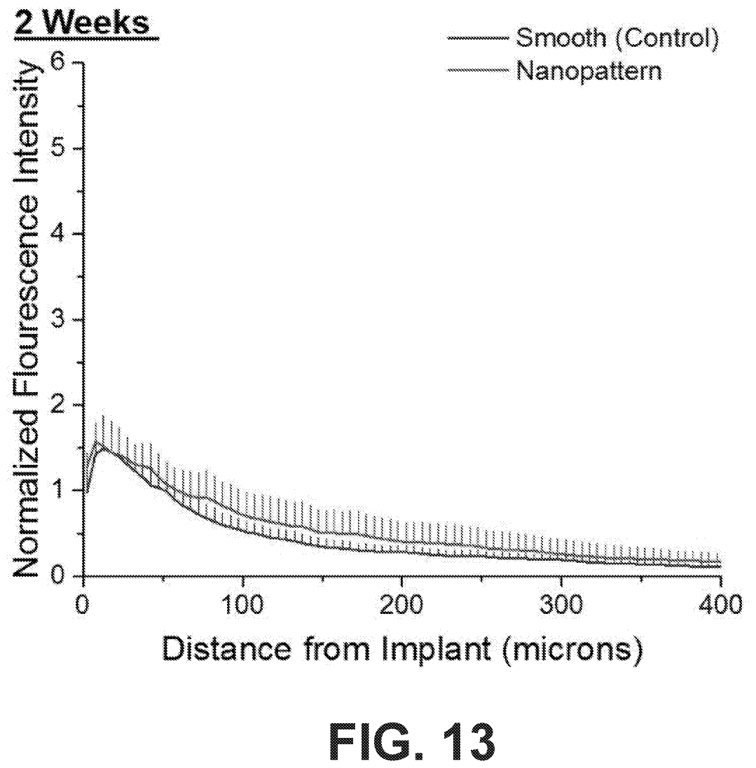

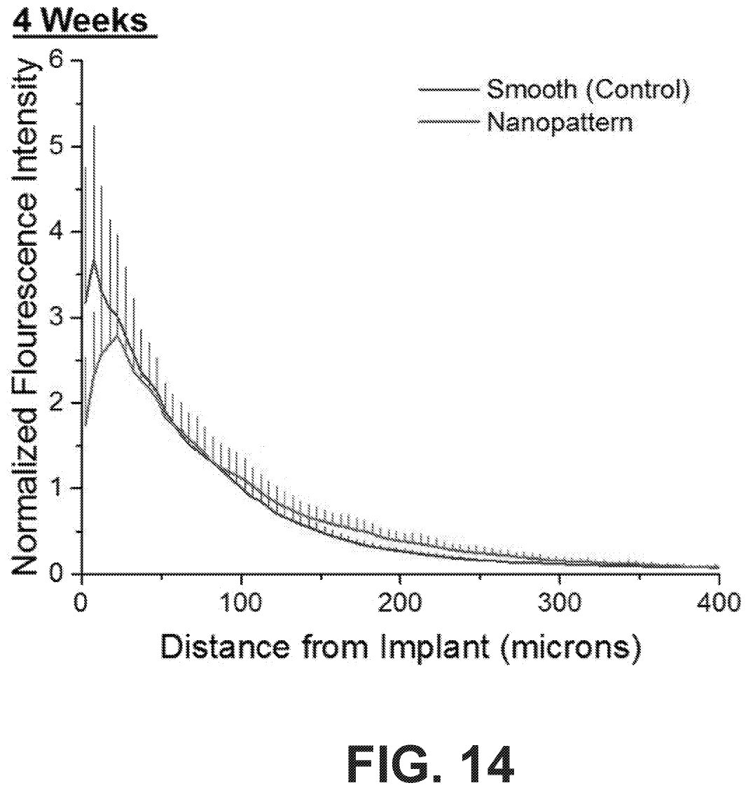

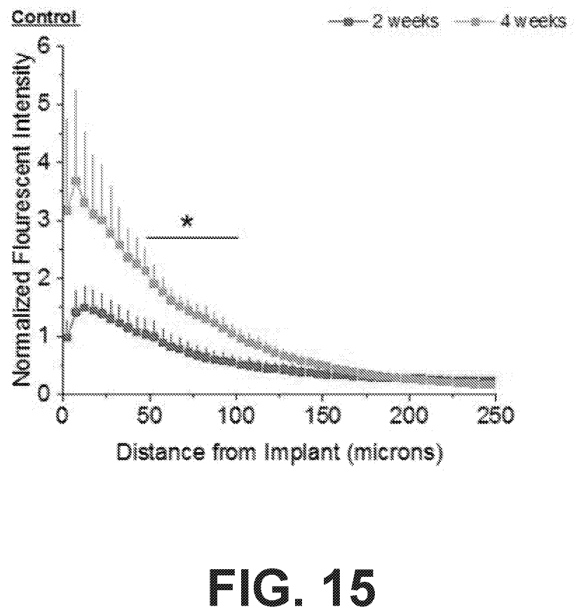

At both two and four weeks post implantation, the levels of IgG around either the nanopatterned or control implant sites were statistically similar (FIGS. 13, 14). There was a significant increase of IgG around the implant sites of the control samples from two to four weeks (p<0.05) (FIG. 15).

The stability of the BBB has become an increasingly important factor to microelectrode performance Histological markers for activated microglia/macrophages and reactive astrocytes were evaluated to correlate with BBB breach and assess the resulting neuroinflammation. No significant differences were found between nanopattern or smooth control groups or over time. However, the trend of decreased neuroinflammation around the nanopatterned implants was evident in the histological assessment indicating a decrease in activated microglia and astrocytes and an increase in neuronal viability over time.

### Effects of Nanopatterned Grooves on Molecular Inflammatory Markers

Although histology proves to be a good technique to identify the cells around the implant site, it is not sensitive enough to predict the phenotype of these cells. The level of sensitivity found with gene expression analysis is much greater than histology, and gives a deeper understanding of the phenotype of the cells, as well as the health, and both neurotoxic and pro-inflammatory cytokines release. Therefore, gene expression analysis was performed to understand the effect nanopatterned grooves have on altering the phenotype of the cells as well as gain insight on the underlying neuroinflammatory mechanisms involved. Specifically, the gene expression of GFAP, interleukin 1 beta (IL1β), tumor necrosis factor-alpha (TNFα), inducible nitric oxide synthase (iNOS/NOS2), and high mobility group box 1 protein (HMGB1) were investigated. GFAP is an astrocyte specific intermediate filament that has increased expression when astrocytes are reactive. IL1β and TNFα are the classic proinflammatory cytokines that are rapidly released during inflammation and primarily involved in the promotion of nuclear factor κ B (NFκB) inflammatory pathway activation. Cluster of differentiation 14 (CD14) is a pattern recognition receptor on the membrane of microglia. CD14 is used to recognize and detect bacteria and pathogens, thus signaling the inflammatory pathway immediately following injury/implantation. High mobility group box 1 protein (HMGB1) is protein that acts like a cytokine when secreted by activated microglia following inflammation, but is also expressed from necrotic cells. In addition to inflammation, oxidative stress markers, such as nitric oxide synthase (NOS2), which is expressed in microglia/macrophages and enhances the synthesis of proinflammatory markers and nitric oxide were observed.

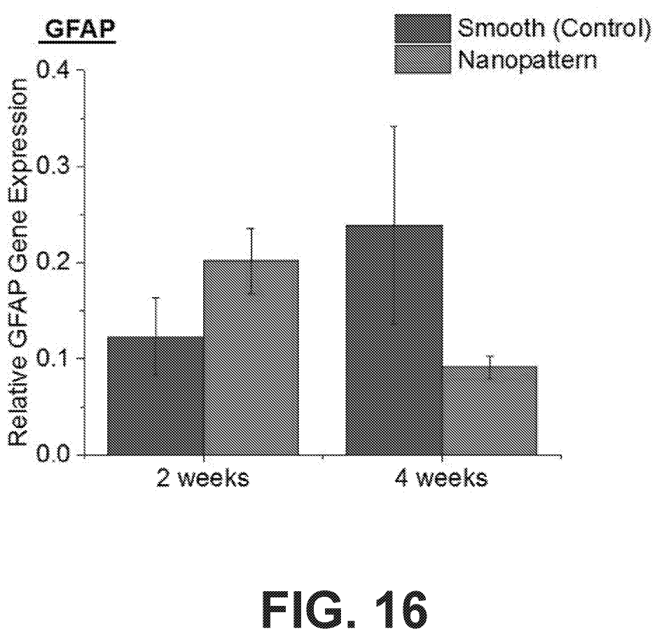

At two and four weeks post implantation, GFAP relative gene expression was not significantly different between animals implanted with nanopatterned or smooth control probes. The observed, non-statistically significant trend of decreased astrocytes around the nanopatterned implant site observed with the histological findings was confirmed with a decrease of GFAP relative gene expression over time as well (FIG. 16). To the contrary, an increase in GFAP relative gene expression over time was observed around the control implants.

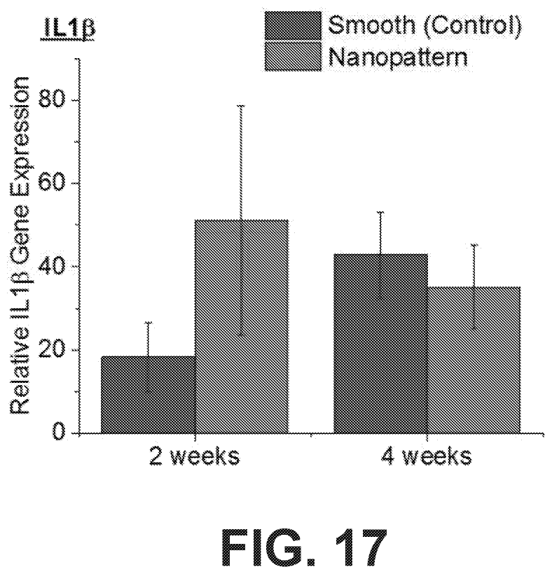

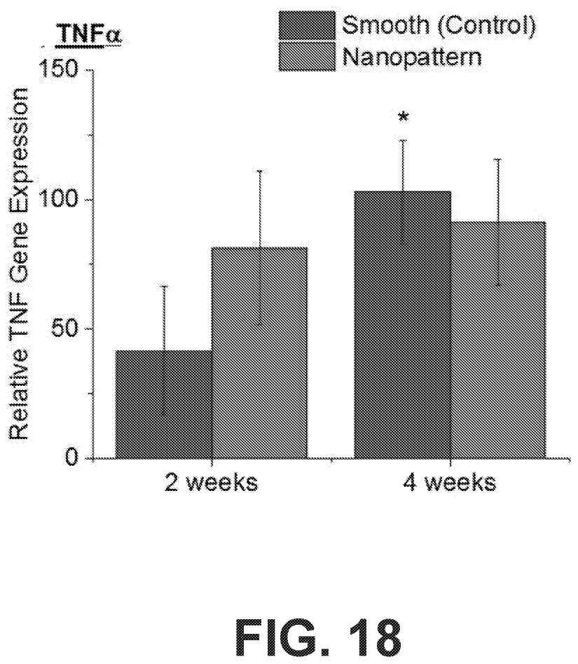

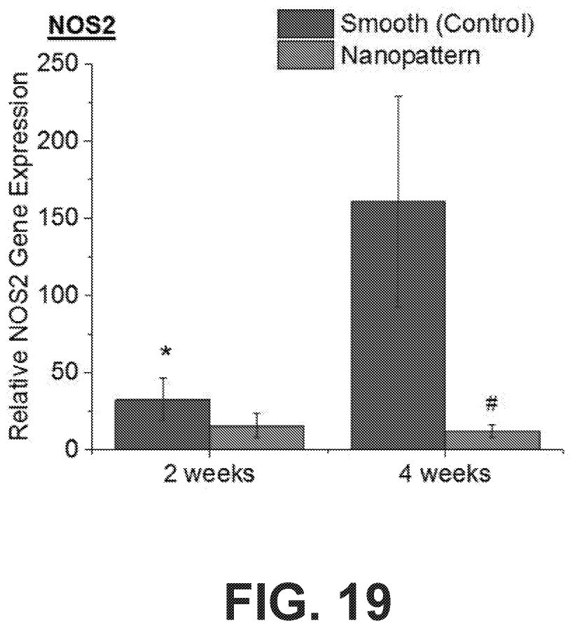

At both two and four weeks post-implantation, the IL1β relative gene expression between nanopatterned and smooth control implant sites were not significantly different. There was a similar observation of increasing IL1β relative gene expression over time around smooth control implants, while a decrease of gene expression occurred around the nanopatterned implants (FIG. 17). There were no significant differences of TNFα relative gene expression found at two or four weeks between nanopattern and smooth control implants (FIG. 18). There were no differences found in TNFα relative gene expression around the nanopattern implant observed over time. However, there was a significant increase (p<0.05) of TNFα relative gene expression from two to four weeks around the smooth control implant site (denoted by * in FIG. 18). At two weeks, there were no significant differences of NOS2 relative gene expression between nanopattern and smooth control implants. However, at four weeks post-implantation, there was significantly less (p<0.05) NOS2 relative gene expression around the nanopattern implants compared to the smooth control implants (denoted by # in FIG. 19). Moreover, there was a significant increase (p<0.05) in NOS2 relative gene expression from two to four weeks around the smooth control implant site (denoted by * in FIG. 19), while there were no differences seen around the nanopattern implants.

The initial breaching of the BBB results in the activation of astrocytes and microglia through the mitogen-activated protein kinase pathway (MAPK). In doing so, levels of IL1β and nitric oxide are both increased. Oxidative stress results in the upregulation of proinflammatory cytokines such as IL1β and TNFα, thereby increasing the neuroinflammatory response. Continuous expression of inflammatory cytokines such as IL-1β can lead to continuous BBB leakage. It is interesting to note, that there were significant increases in IgG, which may indicate disruption of the BBB, around smooth control implants, which correlate with the increase of TNFα and NOS2, over time. Gene expression analysis showed stable levels of TNFα, IL1β, GFAP, and NOS2 from cells around nanopatterned implants. Together, these results may suggest a cease of the self-perpetuating neuroinflammatory pathway previously described for control implants. Furthermore, IL1β in combination with TNFα, have been shown to activate nitric oxide production in astrocytes thereby inducing significant neurotoxicity. The significant increases in TNFα and NOS2 around the smooth control implants indicate possible neurotoxicity around the implant. To the contrary, cells around the nanopatterned implants had significantly lower levels of NOS2 gene expression compared to control implants. These results correlate with the significantly higher levels of neuronal viability around nanopattern implants at four weeks. Although not fully understood, perhaps the nanopatterned grooves inhibit oxidative stress through down regulation of NOS2, which consequently encourages neuronal health and viability.

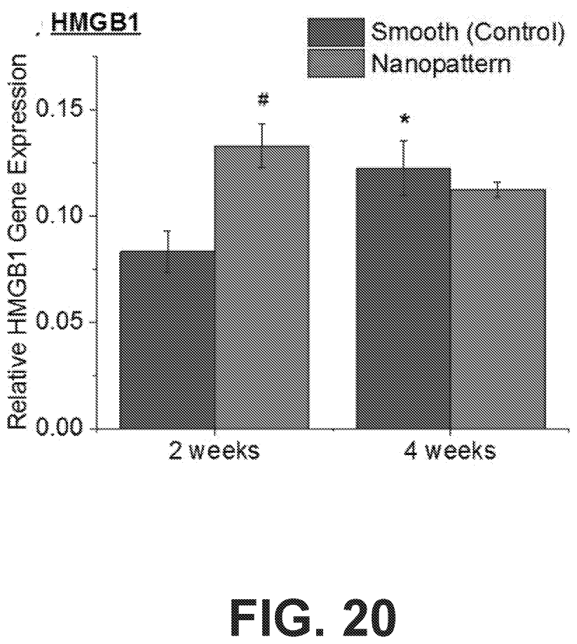

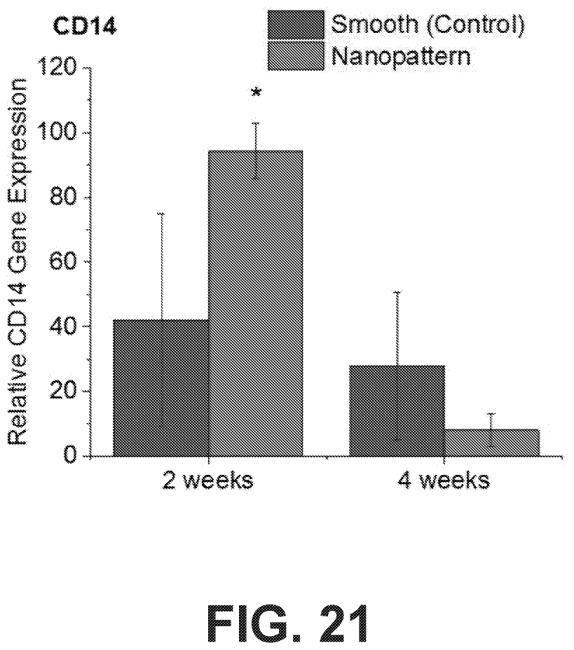

Animals implanted with nanopatterned implants expressed significantly more (p<0.05) HMGB1 relative gene expression compared to smooth control implants at two weeks (denoted by # in FIG. 20). There were no significant differences of relative HMGB1 gene expression observed at four weeks between the two implant types. Furthermore, there was no significant change in relative HMGB1 gene expression around nanopattern implants over time. Conversely, there was a significant increase (p<0.05) in relative HMGB1 gene expression around smooth control implants from two to four weeks (denoted by * in FIG. 20). No significant differences of CD14 relative gene expression between nanopattern and smooth control implants were seen at either two or four weeks post implantation. Nevertheless, it was observed that animals implanted with nanopatterned implants had a significant decrease (p<0.05) of CD14 relative gene expression from two to four weeks (denoted by * in FIG. 21). Hence, it was hypothesized that the initial increase in HMGB1 and CD14 gene expression around nanopatterned probes is actually accelerated wound healing, and not necessarily a foreign body response. One of the initial stages of wound healing inflammation is the observation of increases in glial cells activation and cytokine expression. Extracellular HMGB1, released passively by necrotic cells, actively by macrophages and by some of the injured neurons, acts as a proinflammatory cytokine, and has been thought to play a role in BBB disruption. HMGB1 binds to toll like receptors (TLRs), found on the membrane of activated microglial and macrophage cells. HMGB1 dependent TLR4 activation requires the co-receptor CD14. Therefore, future studies should evaluate the role of TLR4 and/or CD14 inhibition as a means to mitigate microelectrode-induced oxidative stress.

The mechanism explaining how surface modifications can influence the neuroinflammatory response to implanted intracortical microelectrode probes is not fully understood. Topography plays a critical role in influencing cell behavior; in fact, cells have been shown to respond to objects as small as 5 nm in their environment. Surface modifications may help integrate the probe into the brain by reducing the foreign body response. It is likely that adding roughness to the implant alters protein adsorption density and the resulting conformation, causing subsequent changes in downstream cellular behavior/phenotype. Surface chemistry and topography are the primary factors that control the amount, type, and architecture of adsorbed proteins. Cell adhesion is mediated by integrin receptors binding to adsorbed proteins and the initial cell adhesion to a material substrate has been shown to mediate long-term cellular functions and phenotype. Moreover, cellular response is specific to the surface structures. When neurons and fibroblasts were cultured onto surface structured platinum and silicon, they were found to have opposing responses due to the structures. The fibroblasts were inhibited while the neuronal attachment and differentiation was stimulated. This was thought to be due poor adhesion of the fibroblasts to the structured surfaces, which was attributed to the conformation and type of adsorbed protein. Adsorbed ECM proteins serve as adhesion ligands, which integrin receptors within the cell membrane bind to for cell attachment. As regulators of cell structure and behavior, integrins can affect adhesion strength, cell morphology, proliferation, survival and differentiation. Once the ligands are bound to the transmembrane receptors, focal adhesions are formed, which can consequently alter the cytoskeleton of the cell. Thus, substrate topography affects the alignment of the actin filaments and the focal adhesion structures, therefore altering the cell morphology, adhesion, and function. For example, when fibroblasts were seeded onto a nanostructured surface, the first event that took place was the cytoskeleton arrangement. Within the first five minutes post seeding, the actin aggregation occurred, then within 20 minutes the microtubules aligned. Further investigation of specific expression of adhesion proteins, their adsorption, conformation and architecture are required in order to optimize and understand the how surface modifications can reduce neuroinflammation.

### Validation for Nanopatterned Grooves on One Side of Electrode

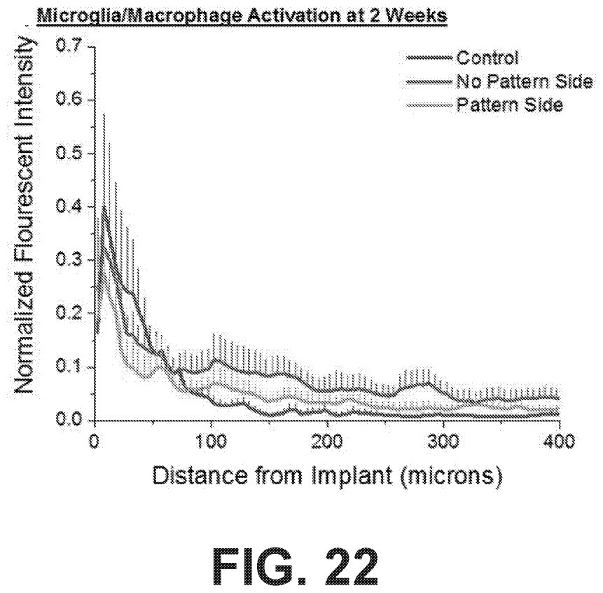

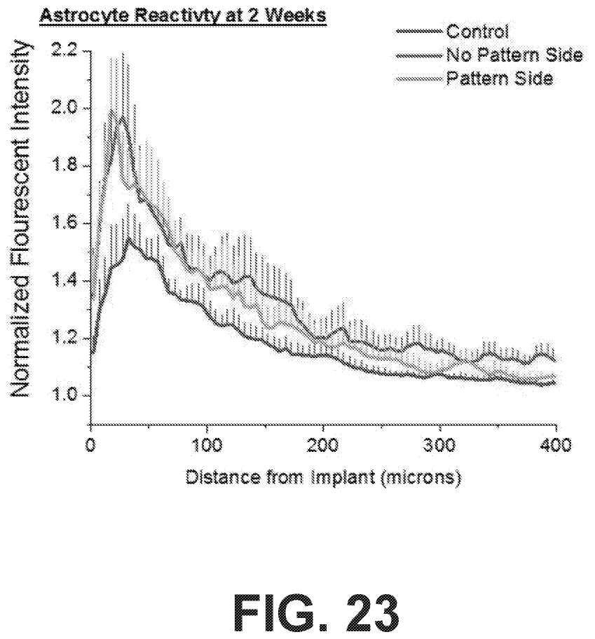

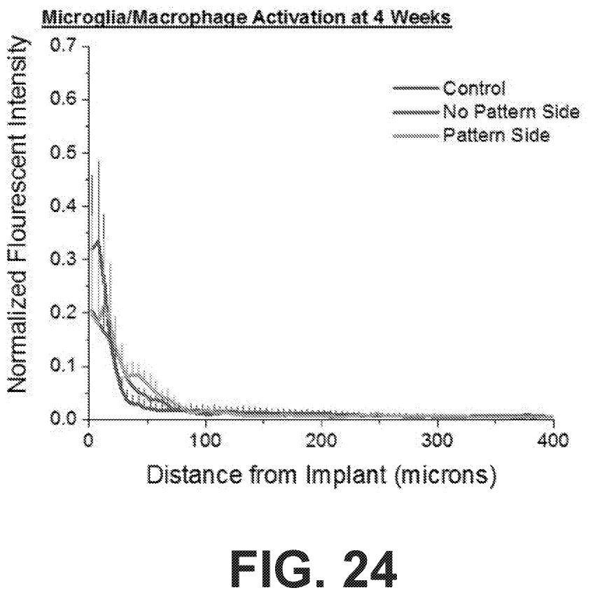

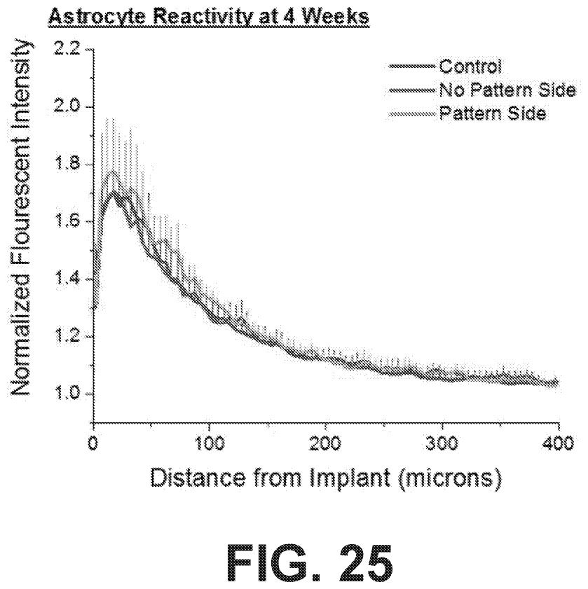

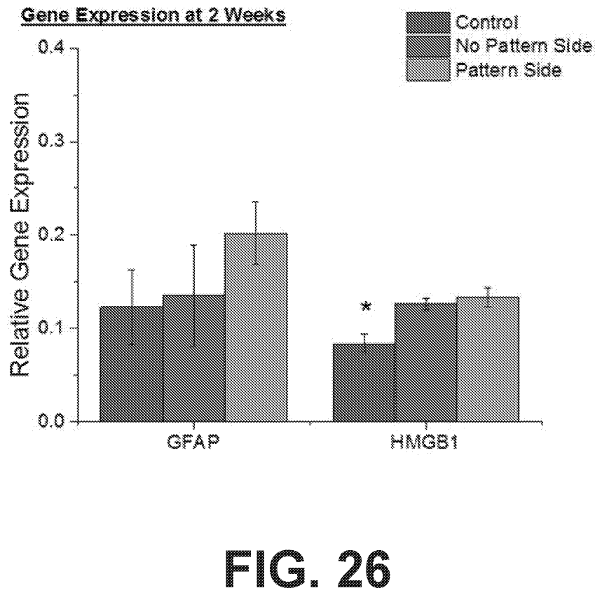

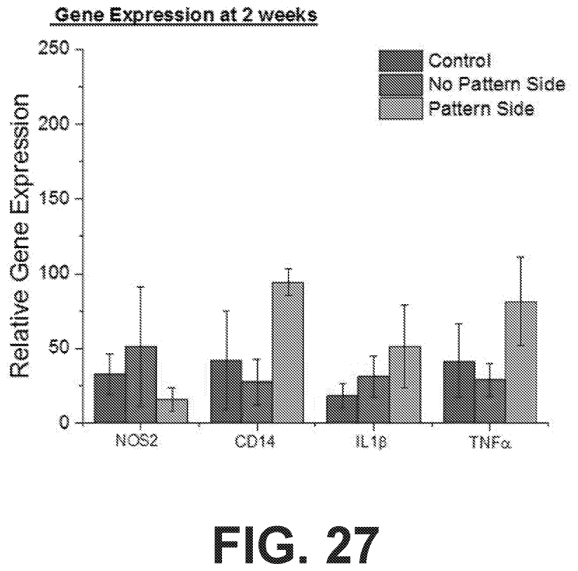

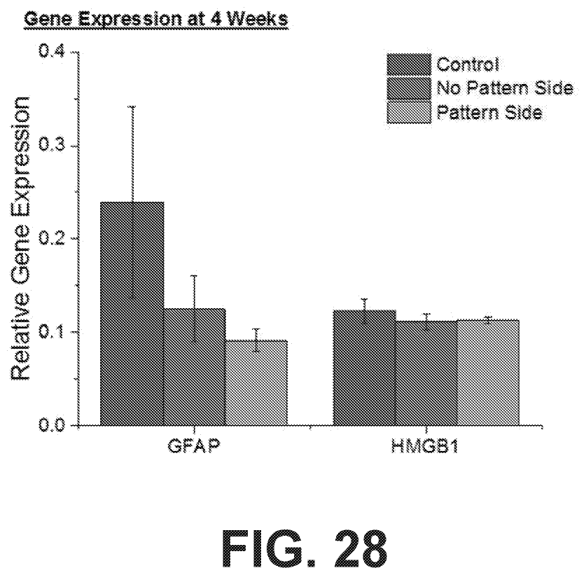

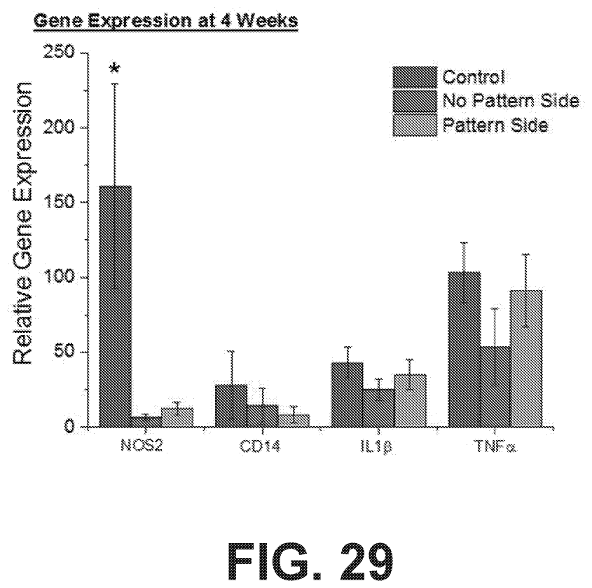

In order to translate the nanopatterned grooves onto functional intracortical microelectrode probes, the cellular and molecular effects from etching nanopatterned grooves onto only one side of the probe were evaluated. Functional Michigan style intracortical microelectrodes consist of recording contact sites and traces along one side of the shank of the microelectrode probe. These recording features pose difficulties in etching nanopatterned grooves along that side of the shank. Therefore, quantification of the fluorescence intensity and the relative gene expression from tissue around the nanopatterned side of the implant were compared to the non-patterned side of the implant. Each side of the nanopatterned implant was then compared the control implants. There were no significant differences found with activated microglia/macrophages, reactive astrocytes, or gene expression of GFAP, HMGB1, NOS2, CD14, IL1β, and TNFα between the nanopatterned side and the non-patterned side of the implants. Furthermore, when comparing to control implants, there were no observed differences of activated microglia (FIGS. 22, 24) or activated astrocytes (FIGS. 23, 25) around the implant site between the control and the nanopatterned or non-patterned side of the implant at two or four weeks. There were no significant differences observed from gene expression of GFAP, CD14, IL1β, and TNFα, between the control and nanopatterned and non-pattered sides of the implant at two or four weeks (FIGS. 26-29). There was significantly more (p<0.05) HMGB1 gene expression around the nanopatterned and non-patterned sides of the implant compared to the control at two weeks (FIG. 26). Conversely, there was significantly less (p<0.05) NOS2 gene expression around the nanopatterned and non-patterned sides of the implant compared to the control at four weeks (FIG. 29).

This study was designed to investigate an efficient translation of nanopatterning onto functional intracortical electrodes without the concern of etching around recording contacts and traces. However, results from this study indicate that future studies should include nanopatterning on both sides, as they may make implications on neuroinflammation and recording performance even more promising.

### Explanted Probe Evaluation

Silicon substrates have been shown to degrade in vivo in a number of studies. Here, evaluation of the explanted probes revealed that the nanopatterned features were still on the probe. Given the resolution of the SEM image obtained after explantation from the animals, it was difficult to determine if silicon oxide is dissolving at a faster rate on nanopatterned surfaces compared to smooth control surface. However, it should be noted that no differences were seen in the adherent cell density as DAPI staining of the cell nuclei on the explanted probes showed nominal cell adhesion to the either microelectrode probe. SEM of explanted control probes were compared to nanopatterned probes as well, and no significant differences were found in cell adhesion and residues on the explanted probes.

From the above description, those skilled in the art will perceive improvements, changes and modifications. Such improvements, changes and modifications are within the skill of one in the art and are intended to be covered by the appended claims.

The following is claimed:

1. A method for manufacturing a medical device, the method comprising:
providing a medical device, wherein the medical device comprises a smooth surface configured to interface with an area of a subject's body; and
modifying the smooth surface of the medical device to have a rough, nanopatterned surface with increased surface area.

2. The method of claim 1, wherein the modifying further comprises etching the smooth surface to create the rough, nanopatterned surface.

3. The method of claim 1, wherein the rough, nanopatterned surface is designed to reduce an immunological foreign body response within the area of the subject's body.

4. The method of claim 1, wherein the rough, nanopatterned surface mimics an architecture or an elastic modulus of the area of the subject's body.

5. The method of claim 1, wherein the rough, nanopatterned surface comprises a plurality of grooves etched into the smooth surface.

6. The method of claim 1, wherein the medical device comprises a microelectrode, a spinal electrode, a shunt, a cuff electrode, or a percutaneous electrode.

7. The method of claim 1, wherein the natural environment of the subject's body comprises a three dimensional meshwork of proteins.

8. The method of claim 7, wherein the nanopatterned surface mimics the three dimensional meshwork of proteins and/or provides additional surface area for the protein structures to bind to.

9. A method comprising:
implanting a medical device into an area of a subject's body,
wherein the medical device comprises a three-dimensional structure with a rough surface configured to interface with the area of the subject's body; and
wherein the rough surface reduces an immune foreign body response to the medical device.

10. The method of claim 9, wherein the rough surface comprises a plurality of grooves etched into a smooth surface of the medical device.

11. The method of claim 10, wherein the natural environment of the subject's body comprises a three dimensional meshwork of protein structures.

12. The method of claim 11, wherein the rough surface mimics the three dimensional meshwork of protein structures and/or provides additional surface area for the protein structures to bind to.

13. The method of claim 9, wherein the medical device comprises a microelectrode, a spinal electrode, a shunt, a cuff electrode, or a percutaneous electrode.

14. The method of claim 9, wherein the area of the subject's body comprises neural tissue.

15. The method of claim 14, wherein the neural tissue comprises central nervous system (CNS) tissue.

16. The method of claim 14, wherein the foreign body response comprises neuroinflammation.

17. A medical device comprising:
a three-dimensional structure configured for implantation into an area of a subject's body,
wherein the three-dimensional structure comprises a rough, nanopatterned surface configured to interface with the area of the subject's body,
wherein the rough, nanopatterned surface mimics a natural environment of the area of the subject's body.

18. The medical device of claim 17, wherein the rough, nanopatterned surface comprises a surface modification that alters at least one of a topology of the surface or an elastic modulus of the surface to mimic the natural environment of the area of the subject's body.

19. The medical device of claim 18, wherein the surface modification comprises an etching in a surface of the three-dimensional structure to create the rough, nanopatterned surface.

20. The medical device of claim 16, wherein the three-dimensional structure comprises a microelectrode, a spinal electrode, a shunt, a cuff electrode, or a percutaneous electrode.
